(19) United States
(12) Patent Application Publication
(10) Pub. No.: US 2023/0303621 A1
(43) Pub. Date: Sep. 28, 2023

(54) IMPROVED PROCESS FOR THE SEPARATION AND ANALYSIS OF PANCREATIC ENZYMES

(71) Applicant: Kashiv BioSciences, LLC, Piscataway, NJ (US)

(72) Inventor: Mukesh Mahajan, Ahmedabad (IN)

(21) Appl. No.: 18/125,616

(22) Filed: Mar. 23, 2023

(30) Foreign Application Priority Data
Mar. 23, 2022 (IN) IN202221016286

(51) Int. Cl. C07K 1/20

(52) U.S. Cl. CPC C07K 1/20

(57) ABSTRACT

The present invention relates to reversed phase ultrahigh-performance liquid chromatography (RP-UHPLC) process by using C18 column at a suitable more than one gradient of mobile phase useful for the separation and characterisation of pancreatic protein mixture comprising at least one enzyme lipase, amylase, protease and variant thereof. The present invention further provides qualitative and quantitative analysis of separated Pancreatic protein mixture suitable for pharmaceutical use.

### FIELD OF THE INVENTION

The present invention relates to reversed phase ultrahigh-performance liquid chromatography (RP-UHPLC) process by using C18 column at a suitable more than one gradient of mobile phase useful for the separation and characterisation of pancreatic protein mixture comprising at least one enzyme lipase, amylase, protease and variant thereof. The present invention further provides qualitative and quantitative analysis of separated Pancreatic protein mixture suitable for pharmaceutical use.

### BACKGROUND OF THE INVENTION

Pancreatic enzymes produced by the body are well known for the integral role they play in the digestion of the foods we eat. Pancreatic juice contains numerous enzymes, including amylase, lipase, protease, carboxyl ester lipase, and phospholipase, and the proenzymes trypsinogen, chymotrypsinogen, and pro-carboxypolypeptidase which are converted in the small intestine to their active forms, trypsin, chymotrypsin, and carboxypeptidase, respectively.

Ongoing efforts to improve the process for separation and analysis of the complex proteins leads this invention to create a new and useful process. Given to the complexities of pancreatic protein, there is a need to apply robust technique to monitor the critical quality attributes of pancreatic protein mixture. The quality assessment is imperative in order to comply with regulatory agency guidelines, one of these attributes is a quantitative assessment of the protein variant(s) present in the pancreatic protein mixture. The presence of undesired protein or quantity of protein in pancreatic protein mixture compromise the safety and efficacy of patient. However, the adequate separation is very important to verify the presence of enzymes and variant thereof in pancreatic protein. Once the adequate separation is achieved of pancreatic protein, it can be further analyzed by suitable techniques known in art like SDS, MALDI TOF, CE-SDS etc. The separation is critical as pancreatic protein mixture is complex protein mixture and comprises various enzymes and variant thereof therefore separation method disclosed in the present application is robust to separate all important proteins which make further analysis more accurate.

The present invention relates to reversed phase ultrahigh-performance liquid chromatography (RP-UHPLC) process with suitable more than one gradient of solvent useful for the separation and characterisation of pancreatic protein mixture. RP-UHPLC separates, analyses, characterizes and quantifies the pancreatin active pharmaceutical agents.

Proteins have multiple hydrophobic moieties to interact with the column. The RP-UHPLC chromatography separates proteins based on their hydrophobic characteristics. Proteins bind to RP-UHPLC columns in aqueous mobile phase and are eluted from the column by increasing the hydrophobicity of mobile phase. The stationary phase is made up of hydrophobic alkyl chains. The present invention provides an improved RP-UHPLC method by using C18 column at a suitable more than one gradient of mobile phase which provides an improved separation of pancreatic protein.

### SUMMARY OF THE INVENTION

In an embodiment, the present invention provides an improved process for the separation, and analysis of pancreatic proteins in a sample of pancreatin protein mixture and species based on their hydrophobicity under reducing and non-reducing condition by using C18, RP-UHPLC column at a suitable gradient of mobile phase.

In an embodiment, the invention provides an improved process by using RP UHPLC C18 column for separating and analysing enzymes in a pancreatin protein mixture wherein the pancreatic enzyme comprises at least lipase, amylase and protease enzyme and variant thereof.

In another embodiment, the present invention provides a process to improve the separation profile of pancreatic protein mixture comprising at least one enzyme amylase, protease and lipase & variant thereof through RP-UHPLC C18 column.

The present invention is designed to develop a precise and rapid analytical RP-UHPLC procedure, which can be used for the separation, and analyzing of pancreatic enzymes in a sample of pancreatin protein mixture.

In an embodiment, the separation and analysis of one or more of pancreatic enzymes and variant thereof present in pancreatic protein mixture comprising:
a) preparing the soluble protein mixture from pancreatic sample;
b) optionally treating the protein mixture with reducing agent;
c) loading the soluble protein mixture onto RP-UHPLC C18 column;
d) treating the RP-UHPLC C18 column with suitable separating solution in mobile phase comprising mobile Phase A and mobile phase B wherein the mobile phase A and mobile phase B comprises organic solvent, wherein the mobile phase A and mobile phase B is performed in more than one gradient; and
e) eluting the one or more pancreatic enzyme and variant thereof.

In an embodiment, the separation and analysis of one or more of pancreatic enzymes and variant thereof present in pancreatic protein mixture comprising:
a) preparing the soluble protein mixture from pancreatic sample;
b) optionally treating the protein mixture with reducing agent;
c) loading the soluble protein mixture onto RP-UHPLC C18 column;
d) treating the RP-UHPLC C18 column with suitable separating solution in mobile phase comprising mobile Phase A and mobile phase B wherein the mobile phase A and mobile phase B comprises organic solvent, wherein the mobile phase A and mobile phase B is performed in more than one gradient; and
e) eluting the one or more pancreatic enzyme and variant thereof, wherein;
   (i) eluting the proteolytic digested products, phospholipase A2 (PLA2) bundles of trypsin, Triacylglycerol lipase, Co-lipase, elastase, carboxyl ester lipase (CEL lipase), Chymotrypsin, Kallikrein glandular and amylase when the concentration of mobile phase B is increased from 0% to about 54%;
   (ii) eluting substantially the carboxypeptidase A1 and carboxypeptidase B when the concentration of mobile phase B is increased from about 36% to about 85%;
   (iii) eluting substantially the formulation components when the concentration of mobile phase B is increased from about 75% to 100%;

In an embodiment, the separation and analysis of one or more of pancreatic enzymes and variant thereof present in pancreatic protein mixture comprising:
a) preparing the soluble protein mixture from pancreatic sample;
b) optionally treating the protein mixture with reducing agent;
c) loading the soluble protein mixture onto RP-UHPLC C18 column;
d) treating the RP-UHPLC C18 column with suitable separating solution in mobile phase comprising mobile Phase A and mobile phase B wherein the mobile phase A and mobile phase B comprises organic solvent, wherein the mobile phase A and mobile phase B is performed in more than one gradient; and
e) eluting the one or more pancreatic enzyme and variant thereof, wherein;
   (i) eluting substantially the proteolytic digested products when the concentration of mobile phase B is increased from 0% to about 30%;
   (ii) eluting substantially phospholipase A2 (PLA2) when the concentration of mobile phase B is increased from about 30% to about 37%;
   (iii) eluting substantially the bundles of trypsin, Triacylglycerol lipase, Co-lipase, chymotrypsin, Kallikrein glandular, carboxyl ester lipase (CEL), elastase and amylase when the concentration of mobile phase B is increased from about 37% to about 45%;
   (iv) eluting substantially the carboxypeptidase A1 and carboxypeptidase B when the concentration of mobile phase B is increased from about 45% to about 75%; and
   (v) eluting substantially the formulation components when the concentration of mobile phase B is increased from about 75% to 100%;

In an embodiment, the separation and analysis of one or more of pancreatic enzymes and variant thereof present in pancreatic protein mixture comprising:
a) preparing the soluble protein mixture from pancreatic sample;
b) optionally treating the protein mixture with reducing agent;
c) loading the soluble protein mixture onto RP-UHPLC C18 column;
d) treating the RP-UHPLC C18 column with suitable separating solution in mobile phase comprising mobile Phase A and mobile phase B wherein the mobile phase A and mobile phase B comprises organic solvent, wherein the mobile phase A and mobile phase B is performed in more than one gradient; and
e) eluting the one or more pancreatic enzyme and variant thereof, wherein;
   (i) eluting substantially the proteolytic digested products when the concentration of mobile phase B is increased from 0% to about 30%, wherein the first gradient elution is performed for about 16 minutes to about 18 minutes;
   (ii) eluting substantially the phospholipase A2 (PLA2) when the concentration of mobile phase B is increased from about 30% to about 37%, wherein the second gradient elution is performed for about 1 minute to about 3 minutes.
   (iii) eluting substantially the bundles of trypsin, Triacylglycerol lipase, chymotrypsin, Kallikrein glandular, carboxyl ester lipase (CEL), elastase, amylase and Co-lipase when the concentration of mobile phase B is increased from about 37% to about 45%, wherein the third gradient elution is performed for about 27 minute to about 33 minutes;
   (iv) eluting substantially the carboxypeptidase A1 and carboxypeptidase B when the concentration of mobile phase B is increased from about 45% to about 75%, wherein the fourth gradient elution is performed for about 13 minutes to about 17 minutes; and
   (v) eluting substantially the formulation components when the concentration of mobile phase B is increased from about 75% to 100%, wherein the fifth gradient elution is performed for about 13 minutes to about 17 minutes.

In an embodiment the invention provides the separation of proteolytic digested products, phospholipase A2 (PLA2) bundles of trypsin, Triacylglycerol lipase, Co-lipase, elastase, carboxyl ester lipase (CEL lipase), Chymotrypsin, Kallikrein glandular and amylase present in pancreatic protein mixture by increasing the concentration of mobile phase B from 0% to about 54% in RP UHPLC C18 column.

In preferred embodiment the invention provides the separation of substantially the proteolytic digested products present in pancreatic protein mixture by increasing the concentration of mobile phase B from 0% to about 30% in RP UHPLC C18 column.

In preferred embodiment the invention provides the separation of substantially phospholipase A2 (PLA2) present in pancreatic protein mixture by increasing the concentration of mobile phase B from about 30% to about 37% in RP UHPLC C18 column.

In preferred embodiment the invention provides the separation of substantially the bundles of trypsin, Triacylglycerol lipase, Co-lipase, chymotrypsin, Kallikrein glandular, carboxyl ester lipase (CEL), elastase and amylase present in pancreatic protein mixture by increasing the concentration of mobile phase B from about 37% to about 45% in RP UHPLC C18 column.

In an embodiment the invention provides the separation of carboxypeptidase A1 and carboxypeptidase B present in pancreatic protein mixture by increasing the concentration of mobile phase B from about 36% to about 85% in RP UHPLC C18 column.

In preferred embodiment the invention provides the separation of carboxypeptidase A1 and carboxypeptidase B present in pancreatic protein mixture by increasing the concentration of mobile phase B from about 45% to about 75% in RP UHPLC C18 column.

In an embodiment the invention provides the separation of substantially the proteolytic digested products present in pancreatic protein mixture by increasing the concentration of mobile phase B from 0% to about 30% in RP UHPLC C18 column wherein the gradient elution is performed for about 18 minutes.

In preferred embodiment the invention provides the separation of substantially phospholipase A2 (PLA2) present in pancreatic protein mixture by increasing the concentration of mobile phase B from about 30% to about 37% in RP UHPLC C18 column.
wherein the second gradient elution is performed for about 20 minutes.

In an embodiment the invention provides the separation of substantially the bundles of trypsin, Triacylglycerol lipase, Co-lipase, chymotrypsin, Kallikrein glandular, carboxyl ester lipase (CEL), elastase and amylase present in pancreatic protein mixture by increasing the concentration of mobile phase B from about 37% to about 45% in RP UHPLC C18 column wherein the gradient elution is performed for about 50 minutes.

In embodiment the invention provides the separation of carboxypeptidase A1 and carboxypeptidase B present in pancreatic protein mixture by increasing the concentration of mobile phase B from about 45% to about 75% in RP UHPLC C18 column.
wherein the gradient elution is performed for about 65 minutes;

In preferred embodiment the invention provides the separation of substantially formulation components present in pancreatic protein mixture by increasing the concentration of mobile phase B from about 75% to about 100% in RP UHPLC C18 column.
wherein the gradient elution is performed for about 80 minutes.

In an embodiment, the present invention provides a process to improve batch to batch consistency to comply with regulatory guideline.

In an embodiment the invention provides pancreatic protein mixture is obtained from crude, partially purified, substantially purified and microbially synthesize.

In an embodiment the invention provides the mobile phase A and mobile phase B comprises organic solvents is selected from iso-propyl alcohol (IPA), acetonitrile (ACN), methanol, trifluoro acetic acid (TFA), formic acid, and mixtures thereof.

In an embodiment the invention provides the mobile phase A is comprising organic solvent 0.1% TFA with water and mobile phase B is comprising organic solvent 0.1% TFA with Acetonitrile.

In an embodiment the invention provides the separated pancreatic proteins and variant thereof are further analysis by one or more suitable technique selected from CE-SDS, SDS-PAGE, MALDI-TOF-MS, MS, SE-HPLC profile and RP-HPLC.

In an embodiment the invention provides the mobile phase A and mobile phase B maintains flow rate from about 0.2 ml/min to about 0.6 ml/min.

In an embodiment the invention provides the separated protein peaks are analyzed by spiking with commercially available standard of pancreatic enzymes, each pancreatic enzymes co-eluting with the separated pancreatic protein peaks identical to it as shown in FIG. 6.

### BRIEF DESCRIPTION OF THE DRAWINGS

FIG. 1: Shows the separation profile of pancreatic proteins through RP-UHPLC using water and 0.05% TFA as mobile phase A and Acetonitrile and 0.05% TFA as mobile phase B at a gradient set as described in the example 1.

FIG. 2: Shows the separation profile of pancreatic proteins under non-reducing conditions by RP-UHPLC using water and 0.1% TFA as mobile phase A and 0.1% TFA and Acetonitrile as mobile phase B at an optimized gradient set as described in example 2.

FIG. 3: Shows the separation profile of pancreatic proteins under reducing conditions by RP-UHPLC using water and 0.1% TFA as mobile phase A and 0.1% TFA and Acetonitrile as mobile phase B at an optimized gradient set as described in example 3.

FIG. 4: Shows the overlay of two pancreatic protein sample chromatograms wherein chromatogram 1 depicts separation of proteolytic digested products along with undigested enzymes present in pancreatic protein mixture and chromatogram 2 depicts the separation undigested enzymes present in pancreatic protein mixture.

FIG. 5: Shows the gradient percentage of mobile phase; a) first gradient, b) second gradient, c) third gradient, d) fourth gradient, and e) fifth gradient.

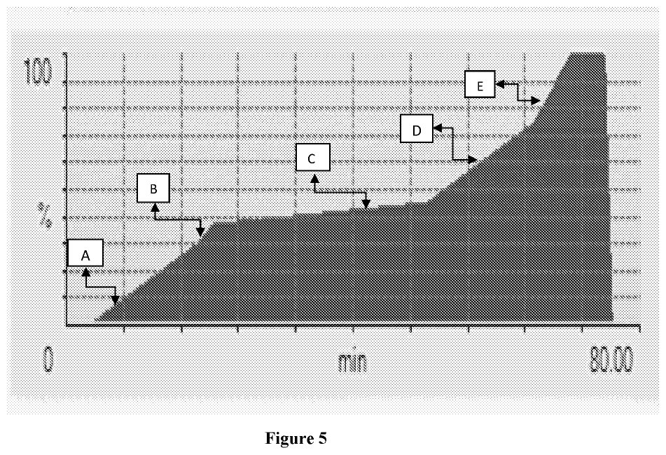

FIG. 6: Shows the chromatogram when the soluble pancreatic protein is extracted from different sources namely a) Standard-native pig elastase-protein, b) Standard-trypsin from porcine pancreas, c) Standard-carboxypeptidase B porcine pancreas, and d) pancreatic protein mixture or samples.

### DETAILED DESCRIPTION OF THE INVENTION

### Definitions

Unless the context clearly requires otherwise, throughout the invention, the words “comprise”, “comprising”, and the like are to be construed in an inclusive sense as opposed to an exclusive or exhaustive sense; that is to say, in the sense of “including, but not limited to”.

The term “about” as used herein is intended to refer to ranges of approximately 5% to 20% greater than or less than the referenced value. In certain circumstances, one skill in the art will recognize that, due to the nature of the referenced value, the term about can mean more or less than 5% to 20% deviation from that value.

The term “substantially” or “substantial” refers to a gradient that substantially elutes the mentioned pancreatic enzymes. The elution of mentioned pancreatic enzymes is about 90% or more, about 91% or more, about 92% or more, about 93% or more, about 94% or more, about 95% or more, about 96% or more, about 97% or more, about 98% or more, about 99% or more, or 100%.

The term “soluble protein mixture” herein refers to the protein sample that is loaded onto RP-UHPLC column. In an embodiment, the “soluble protein mixture” is prepared by first treating pancrelipase granules with suitable buffer selected from citrate phosphate buffer and bicarbonate buffer at pH 6 to about pH 7 or is treated with organic solvent. The prepared sample is thereafter injected with suitable amount of protein before loading the prepared soluble protein mixture on the RP-UHPLC column. In an embodiment, the “soluble protein mixture” is first treated with suitable buffer at pH 6 to about pH 7, followed by treatment with suitable reducing agent, preferably dithiothreitol (DTT). The prepared sample is thereafter injected with suitable amount of protein before loading the prepared soluble protein mixture on the RP-UHPLC column.

The term “gradient” used herein refers to the mixture of solvent A, and solvent B in appropriate concentration and gradually increasing or maintaining the concentration of solvent B with time. In the present disclosure, invention comprises at least one or more than one gradient wherein the first gradient is performed from 0% to about 95% and the second gradient is performed from about 95% to 100%.

In certain embodiment, the invention provides at least one or more than two gradients wherein the first gradient is performed from 0% up to about 95% and the second gradient is performed from about 95% to 100%.

In an embodiment, the invention provides at least one or more than two gradients wherein the first gradient is performed from 0% up to about 30% and the second gradient is performed from about 30% to 37% and the third gradient is performed from about 37% to 45% and the fourth gradient is performed from about 45% to 75% and the fifth gradient is performed from about 75% to 100%.

The term “first gradient” or “first gradient elution” used herein are interchangeable, and herein refers to the percentage of mobile phase B gets completed over a period of time at a particular mixing rate of both mobile phase A and mobile phase B. For example, the first gradient of mobile phase B, gets completed about 30% in 15 minutes which is starting from 3 minutes and completing at about 18 minutes. The rate of completion of mobile phase B is about 2% per minute. The term “first gradient” or “first gradient elution” also refers to the percentage of mobile phase B is from 0% to about 30%.

In an embodiment, the term “second gradient” or “second gradient elution” used herein are interchangeable, and herein refers to another set of mixing rate of both mobile phase A and mobile phase B which is about 3.5% per minute. The term “second gradient” or “second gradient elution” also refers to the percentage of mobile phase B is from about 30% to about 37%.

On the other hand, the term “third gradient” or “third gradient elution” used herein are interchangeable, and herein refers to another set of mixing rate of both mobile phase A and mobile phase B which is about 0.27% per minute. The term “third gradient” or “third gradient elution” also refers to the percentage of mobile phase B is from about 37% to about 45%.

The term “fourth gradient” or “fourth gradient elution” used herein are interchangeable, and herein refers to the mixing rate of both mobile phase A and mobile phase B is about 2% per minute. The term “fourth gradient” or “fourth gradient elution” refers to the percentage of mobile phase B is from about 45% to about 75%.

The term “fifth gradient” or “fifth gradient elution” used herein are interchangeable, and herein refers to the mixing rate of both mobile phase A and mobile phase B is about 2.5% or more per minute. The term “fifth gradient” or “fifth gradient elution” also refers to the percentage of mobile phase B is from about 75% to 100%.

The term “formulation components” used herein refers to the buffer solution comprising excipients, salt variants, etc.

In one embodiment, the invention provides an improved process for the separation and/or analysis of pancreatic protein mixture comprising at least one enzyme amylase, protease, and lipase.

In an embodiment, the separated pancreatic proteins are collected in fractions.

In one embodiment, the quantification can also be performed by method selected from CE-SDS, SDS-PAGE, MALDI-TOF-MS, Mass spectrometry (MS) and SE-HPLC profile.

In an embodiment, the separated pancreatic proteins are further analysed for determining the molecular weights of proteins by CE-SDS or SDS-PAGE technique known in the art.

In an embodiment, the separated pancreatic proteins or fractionated proteins are further characterised by Western Blotting technique known in the art.

In an embodiment, the separated pancreatic proteins or fractionated proteins are further identified by MALDI-TOF-TOF-MS technique known in the art.

In an embodiment, the separation, identification, and analysing of enzymes in a sample of pancreatin protein mixture is performed through RP-UHPLC method.

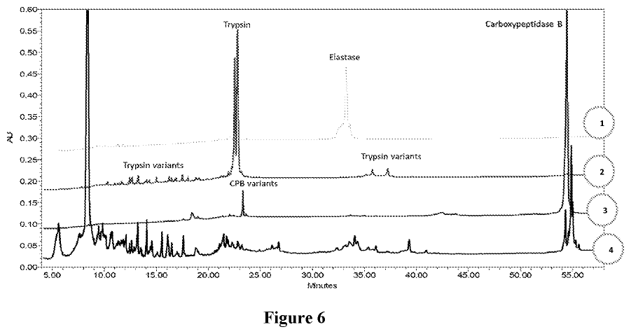

In an embodiment, the separated protein peaks are analyzed by spiking with commercially available standard of pancreatic enzymes, each pancreatic enzymes co-eluting with the separated pancreatic protein peaks identical to it as shown in FIG. 6.

In an embodiment, the process for separation and analysis of one or more of pancreatic enzymes and variant thereof present in pancreatic protein mixture also can be performed through RP-HPLC, preferably RP-UHPLC.

The term “C18” or “C18 column” used herein refers to the stationary phase (resin) of RP-UHPLC which is made up of octadecyl carbon chain (C18)-bonded silica.

The term “pancrelipase samples” or “pancreatic sample” or “pancreatic protein sample”, or “pancreatic enzymes”, or “pancreatic proteins”, are interchangeable with each other and herein refers to pancreatic digestive enzymes formulated in any pharmaceutical composition. In an embodiment, the pancrelipase sample is selected from granules, tablet, capsules, and powder. The “pancrelipase samples” or “pancreatic sample” or “pancreatic protein sample”, or “pancreatic enzymes”, or “pancreatic proteins”, comprises at least one enzyme selected from lipase, protease, amylase, and combination thereof. In an embodiment, the “pancrelipase samples” or “pancreatic sample” or “pancreatic protein sample” obtained from crude, partially purified, substantially purified and microbially synthesize.

In an embodiment, the pancreatic sample contains “enzymes” are selected from Triacylglycerol lipase, Co-lipase, carboxyl ester lipase (CEL lipase), Phospholipase A2, Trypsin, Chymotrypsin, Elastase, Carboxypeptidase A1, Carboxypeptidase B, kallikrein glandular, and Alpha amylase are the prominent functionally important enzymes.

The term “separating solution” refers to water, suitable organic solvent and combination thereof used in mobile phase of RP-UHPLC to separate the pancreatic enzymes present in pancreatic protein mixture based on their hydrophobicity.

The term “organic solvent” refers to solvent selected from Isopropyl alcohol (IPA), acetonitrile (ACN), methanol, trifluoro acetic acid (TFA), formic acid, and mixtures thereof. In certain embodiment, the organic solvent is used in combination with pure water. Organic solvent(s) is used for the preparation of suitable separating solution which is used in mobile phase of RP-UHPLC.

The term “mobile phase” refers to organic solvents which helps to carry the mixture down in the column or eluting proteins from the column. In one aspect, the mobile phase comprises suitable organic solvent and/or water and combination thereof. The term “a suitable mobile phase” refers to mobile phase comprising water, acetonitrile (ACN), trifluoracetic acid (TFA) and combination thereof.

The term “protein variant” or “variant” used herein refers to the fragments of pancreatic proteins or enzymes digested by auto-proteolytic enzymes or post translational modification species e.g., Oxidation, deamidation or isoforms or by using any reducing agents such dithiothreitol (DTT), β-mercaptoethanol (βME) etc. In an embodiment, the protein variant is at least 70% or about 75% or about 80% or about 85% or about 90% or about 91% or about 92% or about 93% or about 94% or about 95% or about 96% or about 97% or about 98% and about 99% identical or similar to protein of interest.

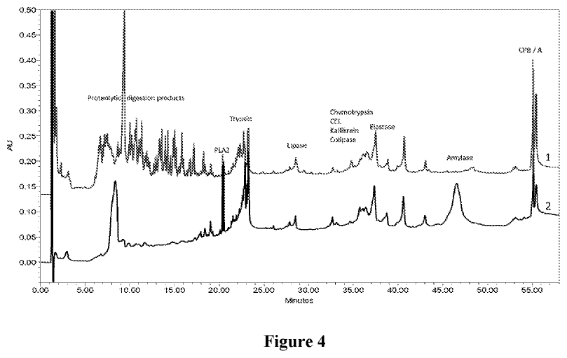

The term “proteolytic digested products” refers to at one or more enzymes digested by the proteolytic enzymes. It also refers to the auto digestion of pancreatic enzymes present in pancreatic sample solution as shown in FIG. 4.

The term “amylase” refers to an enzyme that helps in the chemical process of digestion by hydrolysing starch into more available saccharide forms.

The term “lipase” refers to an enzyme that is secreted by the pancreas and is responsible for the hydrolysis of dietary fat molecules in the human digestive system. Lipases comprises of Triacylglycerol lipase, Co-lipase, Carboxy ester lipase, and phospholipase A2 (PLA 2).

The term “protease” refers to an enzyme that breaks down proteins into smaller polypeptides or amino acids. The protease comprises trypsin, chymotrypsin, elastase, kallikrein glandular, Carboxypeptidase A1, Carboxypeptidase B, and chymotrypsin.

The term “suitable reducing agent” or “reducing agent” refers to a reagent for reducing disulphide bonds between and within biological molecules.

In certain embodiment, the mobile phase comprising a gradient consisting of two mobile phase A and mobile phase B. In an embodiment, mobile phase A comprising TFA, formic acid. In certain embodiment, the mobile phase A further comprising water.

In an embodiment, mobile phase B comprising iso-propyl alcohol (IPA), acetonitrile (ACN), methanol, trifluoro acetic acid (TFA), formic acid, and mixture thereof. In certain embodiment, the mobile phase B further comprising water.

The present invention provides a gradient of mobile phase A and mobile phase B wherein the mobile phase A is gradually decreased, and mobile phase B is gradually increased.

The term “qualitative comparison” and “analytical comparison” refers to visual comparative analysis between reference sample and test sample by means of overlay.

In an embodiment, the present invention provides an improved process for quantification of the protein variants of pancreatic enzymes.

In an embodiment the invention provides the separation of proteolytic digested products, phospholipase A2 (PLA2) bundles of trypsin, Triacylglycerol lipase, Co-lipase, elastase, carboxyl ester lipase (CEL lipase), Chymotrypsin, Kallikrein glandular and amylase present in pancreatic protein mixture by increasing the concentration of mobile phase B from 0% to about 54% in RP UHPLC C18 column.

In preferred embodiment the invention provides the separation of substantially the proteolytic digested products present in pancreatic protein mixture by increasing the concentration of mobile phase B from 0% to about 30% in RP UHPLC C18 column.

In preferred embodiment the invention provides the separation of substantially phospholipase A2 (PLA2) present in pancreatic protein mixture by increasing the concentration of mobile phase B from about 30% to about 37% in RP UHPLC C18 column.

In preferred embodiment the invention provides the separation of substantially the bundles of trypsin, Triacylglycerol lipase, Co-lipase, chymotrypsin, Kallikrein glandular, carboxyl ester lipase (CEL), elastase and amylase present in pancreatic protein mixture by increasing the concentration of mobile phase B from about 37% to about 45% in RP UHPLC C18 column.

In an embodiment the invention provides the separation of carboxypeptidase A1 and carboxypeptidase B present in pancreatic protein mixture by increasing the concentration of mobile phase B from about 36% to about 85% in RP UHPLC C18 column.

In preferred embodiment the invention provides the separation of carboxypeptidase A1 and carboxypeptidase B present in pancreatic protein mixture by increasing the concentration of mobile phase B from about 45% to about 75% in RP UHPLC C18 column.

In an embodiment, the separation and analysis of one or more of pancreatic enzymes and variant thereof present in pancreatic protein mixture comprising:
a) preparing the soluble protein mixture from pancreatic sample;
b) optionally treating the protein mixture with reducing agent;
c) loading the soluble protein mixture onto RP-UHPLC C18 column;
d) treating the RP-UHPLC C18 column with suitable separating solution in mobile phase comprising mobile Phase A and mobile phase B wherein the mobile phase A and mobile phase B comprises organic solvent, wherein the mobile phase A and mobile phase B is performed in more than one gradient; and
e) eluting the one or more pancreatic enzyme and variant thereof.

In an embodiment, the separation and analysis of one or more of pancreatic enzymes and variant thereof present in pancreatic protein mixture comprising:
a) preparing the soluble protein mixture from pancreatic sample;
b) optionally treating the protein mixture with reducing agent;
c) loading the soluble protein mixture onto RP-UHPLC C18 column;
d) treating the RP-UHPLC C18 column with suitable separating solution in mobile phase comprising mobile Phase A and mobile phase B wherein the mobile phase A and mobile phase B comprises organic solvent, wherein the mobile phase A and mobile phase B is performed in more than one gradient; and
e) eluting the one or more pancreatic enzyme and variant thereof, wherein;
   (i) eluting the proteolytic digested products, phospholipase A2 (PLA2) bundles of trypsin, Triacylglycerol lipase, Co-lipase, elastase, carboxyl ester lipase (CEL lipase), Chymotrypsin, Kallikrein glandular and amylase when the concentration of mobile phase B is increased from about 0% to about 54%;
   (ii) eluting substantially the carboxypeptidase A1 and carboxypeptidase B when the concentration of mobile phase B is increased from about 36% to about 85%;
   (iii) eluting substantially the formulation components when the concentration of mobile phase B is increased from about 75% to 100%.

In an embodiment, the separation and analysis of one or more of pancreatic enzymes and variant thereof present in pancreatic protein mixture comprising:
a) preparing the soluble protein mixture from pancreatic sample;
b) optionally treating the protein mixture with reducing agent;
c) loading the soluble protein mixture onto RP-UHPLC C18 column;
d) treating the RP-UHPLC C18 column with suitable separating solution in mobile phase comprising mobile Phase A and mobile phase B wherein the mobile phase A and mobile phase B comprises organic solvent, wherein the mobile phase A and mobile phase B is performed in more than one gradient; and
e) eluting the one or more pancreatic enzyme and variant thereof, wherein;
   (i) eluting substantially the proteolytic digested products when the concentration of mobile phase B is increased from 0% to about 30%;
   (ii) eluting substantially the phospholipase A2 (PLA2) when the concentration of mobile phase B is increased from 30% to about 37%;
   (iii) eluting substantially the bundles of trypsin, Triacylglycerol lipase, Co-lipase, elastase, carboxyl ester lipase (CEL lipase), Chymotrypsin, Kallikrein glandular, and amylase when the concentration of mobile phase B is increased from about 37% to about 45%;
   (iv) eluting substantially the carboxypeptidase A1 and carboxypeptidase B when the concentration of mobile phase B is increased from about 45% to about 75%; and
   (v) eluting substantially the formulation components when the concentration of mobile phase B is increased from about 75% to 100%;

In an embodiment, the separation and analysis of one or more of pancreatic enzymes and variant thereof present in pancreatic protein mixture comprising:
a) preparing the soluble protein mixture from pancreatic sample;
b) optionally treating the protein mixture with reducing agent;
c) loading the soluble protein mixture onto RP-UHPLC C18 column;
d) treating the RP-UHPLC C18 column with suitable separating solution in mobile phase comprising mobile Phase A and mobile phase B wherein the mobile phase A and mobile phase B comprises organic solvent, wherein the mobile phase A and mobile phase B is performed in more than one gradient; and
e) eluting the one or more pancreatic enzyme and variant thereof, wherein;
   (i) eluting substantially the proteolytic digested products when the concentration of mobile phase B is increased from 0% to about 30%, wherein the first gradient elution is performed for about 16 minutes to about 18 minutes;
   (ii) eluting substantially phospholipase A2 (PLA 2) products when the concentration of mobile phase B is increased from about 30% to about 37%, wherein the second gradient elution is performed for about 1 minute to about 3 minutes;
   (iii) eluting substantially the bundles of trypsin, Triacylglycerol lipase, Co-lipase, elastase, carboxyl ester lipase (CEL lipase), Chymotrypsin, Kallikrein glandular, and amylase when the concentration of mobile phase B is increased from about 37% to about 45%, wherein the third gradient elution is performed for about 27 minute to about 33 minutes;
   (iv) (iv) eluting substantially the carboxypeptidase A1 and carboxypeptidase B when the concentration of mobile phase B is increased from about 45% to about 75%, wherein the fourth gradient elution is performed for about 13 minutes to about 17 minutes;
   (v) (v) eluting substantially the formulation components when the concentration of mobile phase B is increased from about 75% to 100%, wherein the fourth gradient elution is performed for about 13 minutes to about 17 minutes.

In an embodiment, the proteolytic digested products are eluted when the concentration of the mobile phase B is increased from about 24%, about 25%, about 26%, about 27%, about 28%, about 29%, about 30%, about 31%, about 32%, about 33%, about 34%, about 35%, and about 36%.

In an embodiment, the proteolytic digested products are eluted when the concentration of the mobile phase B is increased from about 27%, about 28%, about 29%, about 30%, about 31%, about 32%, and about 33%.

In another embodiment, the proteolytic digested products are eluted when the concentration of the mobile phase B is increased from about 29%, about 30%, and about 31%.

In another embodiment, the proteolytic digested products are eluted when the concentration of the mobile phase B is increased at about 30%.

In an embodiment, the phospholipase A2 (PLA 2) are eluted when the concentration of the mobile phase B is increased from about 30%, about 31%, about 32%, about 33%, about 34%, about 35%, about 36%, about 36%, about 37%, about 38%, about 39%, about 40%, about 41%, about 42%, and about 43%.

In an embodiment, the phospholipase A2 (PLA 2) are eluted when the concentration of the mobile phase B is increased from, about 34%, about 35%, about 36%, about 37%, about 38%, about 39%, and about 40%.

In an embodiment, the phospholipase A2 (PLA 2) are eluted when the concentration of the mobile phase B is increased from about 36%, about 37%, and about 38%.

In an embodiment, the phospholipase A2 (PLA 2) are eluted when the concentration of the mobile phase B is increased from at about 37%.

In an embodiment, bundles of trypsin, Triacylglycerol lipase, Co-lipase, elastase, carboxyl ester lipase (CEL lipase), Chymotrypsin, Kallikrein glandular, and amylase are eluted when the concentration of mobile phase B is increased from about 30%, about 31%, about 32% about 33%, about 34%, about 35, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, and about 54%.

In an embodiment, bundles of trypsin, Triacylglycerol lipase, Co-lipase, elastase, carboxyl ester lipase (CEL lipase), Chymotrypsin, Kallikrein glandular, and amylase are eluted when the concentration of mobile phase B is increased from about 33%, about 34%, about 35%, about 36%, about 37%, about 38, about 39%, about 40%, about 41%, about 42%, about 43%, about 44%, about 45%, about 46%, about 47%, about 48%, and about 49%.

In an embodiment, bundles of trypsin, Triacylglycerol lipase, Co-lipase, elastase, carboxyl ester lipase (CEL lipase), Chymotrypsin, Kallikrein glandular, and amylase are eluted when the concentration of mobile phase B is increased from about 35%, about 36%, about 37%, about 38%, about 39%, about 40, about 41%, about 42%, about 43%, about 44%, about 45%, about 46%, and about 47%.

In an embodiment, trypsin Triacylglycerol lipase and Co-lipase bundles of trypsin, Triacylglycerol lipase, Co-lipase, elastase, carboxyl ester lipase (CEL lipase), Chymotrypsin, Kallikrein glandular, and amylase are eluted when the concentration of mobile phase B is increased from about 37%, to about 45%.

In an embodiment, carboxypeptidase A1 and carboxypeptidase B are eluted when the concentration of mobile phase B is increased from about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54% about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79% about 80%, about 81%, about 82%, about 83%, about 84%, about 85%, about 86%, about 87%, about 88%, about 89%, and about 90%.

In an embodiment, carboxypeptidase A1 and carboxypeptidase B are eluted when the concentration of mobile phase B is increased from about 41%, about 42%, about 43%, about 44%, about 45%, about 46%, about 47%, about 48%, about 49%, about 50%, about 51%, about 52%, about 53%, about 54% about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79% about 80%, about 81%, and about 82%.

In an embodiment, carboxypeptidase A1 and carboxypeptidase B are eluted when the concentration of mobile phase B is increased from about 41%, about 42%, about 43%, about 44%, about 45%, about 46%, about 47%, about 48%, about 49%, about 50%, about 51%, about 52%, about 53%, about 54% about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, and about 78%.

In an embodiment, carboxypeptidase A1 and carboxypeptidase B are eluted when the concentration of mobile phase B is increased from about 45% to about 75%.

In an embodiment, formulation components is eluted when the concentration of mobile phase B is increased from about 70%, about 71%, about 72%, about 73%, about 74%, about 75%, about 76%, about 77%, about 78%, about 79% about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, and 100%.

In an embodiment, eluting substantially the proteolytic digested products when the concentration of mobile phase B is increased from 0% to about 30%, wherein the first gradient elution is performed for about 18 minutes.

In an embodiment, eluting substantially the phospholipase A2 (PLA 2) when the concentration of mobile phase B is increased from about 30% to about 37%, wherein the first gradient elution is performed for about 2 minutes.

In an embodiment, eluting substantially the bundles of trypsin, Triacylglycerol lipase, Co-lipase, elastase, carboxyl ester lipase (CEL lipase), Chymotrypsin, Kallikrein glandular, and amylase when the concentration of mobile phase B is increased from about 37% to about 45%, wherein the second gradient elution is performed for about 30 minutes.

In an embodiment, eluting substantially the carboxypeptidase A1 and carboxypeptidase B when the concentration of mobile phase B is increased from about 45% to about 75%, wherein the fourth gradient elution is performed for about 15 minutes.

In an embodiment, eluting substantially the formulation components when the concentration of mobile phase B is increased from about 75% to 100%, wherein the fourth gradient elution is performed for about 15 minutes.

In an embodiment, the reducing agent is selected from β-mercaptoethanol (β-MCE), and tris(2-carboxyethyl) phosphine (TCEP) and most preferably dithiothreitol (DTT). In an embodiment, the concentration of reducing agent (DTT) is 1M.

In an embodiment, the detection is carried out at UV frequency 214 nm.

The gradient separates the major protein peak within 70 minutes, preferably about 60 minutes and most preferably about 52 minutes.

In an embodiment, the flow rate is maintained from about 0.1 ml/min, about 0.2 ml/min, about 0.3 ml/min, about 0.4 ml/min, about 0.5 ml/min, about 0.6 ml/min, ml/min, about 0.7 ml/min, about 0.8 ml/min, about 0.9 ml/min and about 1 ml/min.

In certain embodiment, the flow rate is maintained at about 0.3 ml/min.

In an embodiment, the RP-UHPLC column temperature is maintained from about 60° C., about 65° C., about 70° C., about 75° C. and about 80° C.

In an embodiment, the column temperature is maintained at about 70° C.

In an embodiment, the RP-UHPLC column used herein is a C18 column (2.1X150 mm, 1.7 μm, 300 Å).

In an embodiment the sample temperature is maintained at about 5° C., 6° C., 7° C., 8° C., 9° C.

In an embodiment, the sample temperature is maintained at about 7° C.

The present invention provides below examples for illustrative purpose only and invention should not be considered limiting to below examples.

### Example 1

RP-HPLC C18 Column:

The pancreatic protein sample was injected with 20 μL of sample volume. The separation is performed by using reverse phase C-18 column (4.6 X150 mm, 3 μm, 300 Å) with gradient of acetonitrile as shown in Table 1, detected by UV at 214 nm. Flow rate was maintained at 1.0 mL/min. The column temperature maintained at 25° C. and sample temperature kept at 5° C. Sample run in the system 80 minutes. mobile phase A is 0.05 TFA in Water and mobile phase B is 0.05% TFA in Acetonitrile. Needle washed with 5% (v/v) methanol in water.

TABLE 1
Gradient parameters of RP-UHPLC

| | Mobile Phase A | Mobile Phase B |
|---|---|---|
| Time (min) | (%) | (%) |
| 0.0 | 100 | 0 |
| 75.0 | 0 | 90 |
| 75.1 | 100 | 0 |
| 80 | 100 | 0 |

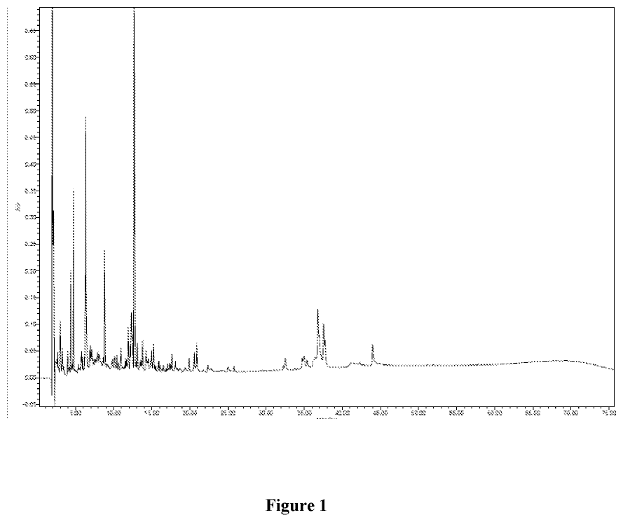

It is evident from FIG. 1 that a linear gradient (a linear gradient of 0 to 90% ACN) process was not able to separate desired enzyme peaks of pancreatic protein mixture (peaks between 30 to 40 min).

### Example 2

RP-UHPLC C18 Column:

For non-reduce analysis, pancreatic protein sample was injected with 20 μg of protein amount. From this non-reduced sample injected 20 μg protein amount. The separation achieved by using reverse phase C18 column (2.1X150 mm, 1.7 μm, 300 Å) with gradient of acetonitrile as shown in Table 2, detected by UV at 214 nm. Flow rate was maintained at 0.3 mL/min. The column temperature maintained at 70° C. and sample temperature kept at 7° C. Sample run in the system 80 minutes. mobile phase A is 0.1% TFA in Water and mobile phase B is 0.1% TFA in Acetonitrile. Needle washed with 5% (v/v) methanol in water.

TABLE 2
Gradient parameters of RP-UHPLC

| | Mobile Phase A | Mobile Phase B |
|---|---|---|
| Time (min) | (%) | (%) |
| 0.0 | 100 | 0 |
| 3.0 | 100 | 0 |
| 18.0 | 70 | 30 |
| 20.0 | 63 | 37 |
| 50.0 | 55 | 45 |
| 65.0 | 25 | 75 |
| 70.0 | 0 | 100 |
| 75.0 | 0 | 100 |
| 76.0 | 100 | 0 |
| 80.0 | 100 | 0 |

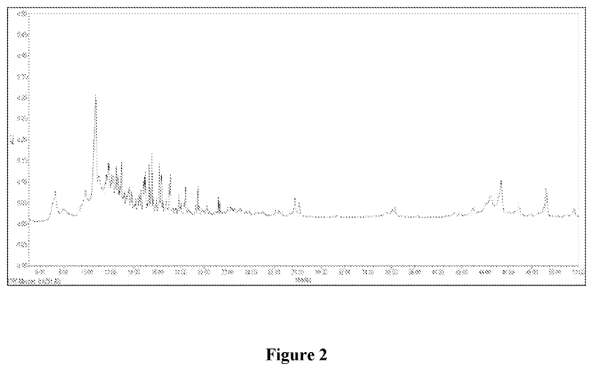

It is evident from FIG. 2 that proteins present in pancreatic protein sample mixture is well separated under non-reducing conditions by RP-UHPLC.

### Example 3

RP-UHPLC C18 Column:

For reducing analysis, pancreatic protein sample was reduced with DTT, wherein the final concentration of DTT in sample was 10 mM. For this reduction process, added 4 μL of 1M DTT solution into 396 μL test sample (concentration 1 mg/mL). Incubated at 37° C. for 30 minutes. The separation achieved by using reverse phase C18 column (2.1X150 mm, 1.7 μm, 300 Å) with gradient of acetonitrile as shown in Table 2, detected by UV at 214 nm. Flow rate was maintained at 0.3 mL/min. The column temperature maintained at 70° C. and sample temperature kept at 7° C. Sample run in the system 80 minutes. mobile phase A is 0.1% TFA in Water and mobile phase B is 0.1% TFA in Acetonitrile. Needle washed with 5% (v/v) methanol in water.

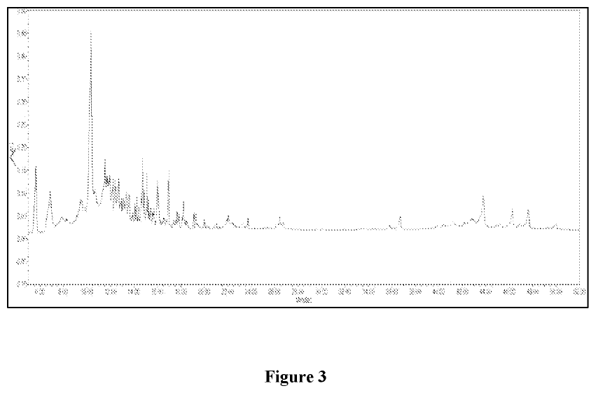

It is evident from FIG. 3 that proteins present in pancreatic protein sample mixture is well separated under reducing conditions by RP-UHPLC.

### Example 4

RP-HPLC C18:

The pancreatic protein sample or mixture is injected with 20 μg of protein amount. From this non-reduced sample injected 20 μg protein amount. The separation is achieved by using reverse phase C18 column 2.1X150 mm, 1.7 μm, 300 Å) with gradient of acetonitrile as shown in Table 2, detected by UV at 214 nm. Flow rate is maintained at 0.3 mL/min. The column temperature is maintained at 65° C. and sample temperature kept at 7° C. Sample run in the system is 80 minutes. mobile phase A is 0.1% TFA in Water and mobile phase B is 0.1% TFA in Acetonitrile. Needle is washed with 5% (v/v) methanol in water.

The pancreatic proteins are separated and analysed for further evaluation.

We claim:

1. An improved process for the separation and analysis of one or more of pancreatic enzymes and variant thereof present in pancreatic protein mixture comprising:
a) preparing the soluble protein mixture from pancreatic sample;
b) optionally treating the protein mixture with reducing agent;
c) loading the soluble protein mixture onto RP-UHPLC C18 column;
d) treating the RP-UHPLC C18 column with suitable separating solution in mobile phase comprising mobile Phase A and mobile phase B wherein the mobile phase A and mobile phase B comprises organic solvent, wherein the mobile phase A and mobile phase B is performed in more than one gradient; and
e) eluting the one or more pancreatic enzyme and variant thereof.

2. The process as claimed in claim 1, wherein eluting the one or more pancreatic enzyme and variant thereof, wherein;
(i) eluting the proteolytic digested products, phospholipase A2 (PLA2) bundles of trypsin, Triacylglycerol lipase, Co-lipase, elastase, carboxyl ester lipase (CEL lipase), Chymotrypsin, Kallikrien glandular and amylase when the concentration of mobile phase B is increased from 0% to about 54%;
(ii) eluting substantially the carboxypeptidase A1 and carboxypeptidase B when the concentration of mobile phase B is increased from about 36% to about 85%;
(iii) eluting substantially the formulation components when the concentration of mobile phase B is increased from about 75% to 100%;

3. The process as claimed in claim 2, wherein eluting the one or more pancreatic enzyme and variant thereof, wherein;
(i) eluting substantially the proteolytic digested products when the concentration of mobile phase B is increased from 0% to about 30%;
(ii) eluting substantially phospholipase A2 (PLA2) when the concentration of mobile phase B is increased from about 30% to about 37%;
(iii) eluting substantially the bundles of trypsin, Triacylglycerol lipase, Co-lipase, chymotrypsin, kallikrein glandular, carboxyl ester lipase (CEL), elastase and amylase when the concentration of mobile phase B is increased from about 37% to about 45%;
(iv) eluting substantially the carboxypeptidase A1 and carboxypeptidase B when the concentration of mobile phase B is increased from about 45% to about 75%;
(v) eluting substantially the formulation components when the concentration of mobile phase B is increased from about 75% to 100%;

4. The process as claimed in claim 1, wherein the pancreatic protein mixture is obtained from crude, partially purified, substantially purified and microbially synthesize.

5. The process as claimed in claim 1, wherein the mobile phase A and mobile phase B comprises organic solvents is selected from iso-propyl alcohol (IPA), acetonitrile (ACN), methanol, trifluoro acetic acid (TFA), formic acid, and mixtures thereof.

6. The process as claimed in claim 5, wherein the mobile phase A is comprising organic solvent 0.1% TFA with water and mobile phase B is comprising organic solvent 0.1% TFA with Acetonitrile.

7. The process as claimed in claim 1, wherein the separated pancreatic proteins and variant thereof are further analysis by one or more suitable technique selected from CE-SDS, SDS-PAGE, MALDI-TOF-MS, MS, SE-HPLC profile and RP-HPLC.

8. The process as claimed in claim 3, wherein the step (i) is performed for about 16 minutes to about 18 minutes; wherein the step (ii) is performed for about 1 minute to about 3 minutes; wherein the step (iii) is performed for about 27 minutes to about 33 minutes; wherein the step (iv) is performed for about 13 minutes to about 17 minutes; wherein the step (v) is performed for about 13 minutes to about 17 minutes.

9. The process as claimed in claim 8, wherein the step (i) is performed for about 18 minutes; wherein the step (ii) is performed for about 2 minutes; wherein the step (iii) is performed for about 30 minutes; wherein the step (iv) is performed for about 15 minutes; wherein the step (v) is performed for about 15 minutes.

10. The process as claimed in claim 1, wherein the mobile phase A and mobile phase B maintains flow rate from about 0.2 ml/min to about 0.6 ml/min.

11. The process as claimed in claim 3, the separated protein peaks are analyzed by spiking with commercially available standard of pancreatic enzymes, each pancreatic enzymes co-eluting with the separated pancreatic protein peaks identical to it as shown in FIG. 6.
